(19) United States
(12) Patent Application Publication
(10) Pub. No.: US 2024/0390316 A1
(43) Pub. Date: Nov. 28, 2024

(54) USE OF DOXEPIN HYDROCHLORIDE IN PREPARATION OF ANTIVIRAL DRUG

(71) Applicant: SHENZHEN TECHNOLOGY UNIVERSITY, Shenzhen (CN)

(72) Inventors: Qinchang Zhu, Shenzhen (CN); Yongqi Yang, Shenzhen (CN); Ge Liu, Shenzhen (CN); Zhendan He, Shenzhen (CN)

(21) Appl. No.: 18/780,670

(22) Filed: Jul. 23, 2024

Related U.S. Application Data
(63) Continuation of application No. PCT/CN2022/140875, filed on Dec. 22, 2022.

(30) Foreign Application Priority Data
Jan. 25, 2022 (CN) 202210086893.0

(51) Int. Cl. A61K 31/335; A61P 31/14

(52) U.S. Cl. CPC A61K 31/335; A61P 31/14

(57) ABSTRACT

The present disclosure provides use of doxepin hydrochloride in preparation of an antiviral drug and relates to the technical field of medicine. Studies show that the doxepin hydrochloride exhibits strong antiviral activity against Coxsackievirus B type 1, type 2 and type 3 and has a 50% minimal inhibitory concentration (IC₅₀) value of 10.12±0.85 μM. In addition, it is also found that the doxepin hydrochloride inhibits virus replication at an early stage of an infection cycle, rather than affecting an entry or assembly process. Meanwhile, some host genes associated with a mechanism are found through target prediction and gene association network analysis. The above findings provide a foundation and a basis for use of the doxepin hydrochloride in treatment of Coxsackievirus B infection.

### CROSS-REFERENCE TO RELATED APPLICATIONS

The present application is a continuation application of PCT application No. PCT/CN2022/140875 filed on Dec. 22, 2022, which claims the benefit of Chinese Patent Application No. 202210086893.0 filed on Jan. 25, 2022. The contents of all of the aforementioned applications are incorporated by reference herein in their entirety.

### TECHNICAL FIELD

The present disclosure relates to the technical field of medicine and specifically relates to use of doxepin hydrochloride in preparation of an antiviral drug.

### BACKGROUND

A Coxsackievirus B (CVB) is a subgroup of a human enterovirus B (HEV-B), which belongs to the Parvoviridae and includes six serotypes: B1 to B6. The CVB is usually considered as a pathogen of human heart diseases and muscle diseases, especially myocarditis. In addition, it has been reported that other inflammatory diseases, such as aseptic meningitis, pleuralgia, panuveitis, acute pancreatitis, and severe hepatitis, are associated with CVBs infection. In recent years, some evidence shows that the CVB can also lead to hand-foot-and-mouth disease (HFMD). Common pathogenic subtypes include B3 and B5, which have related medical cases in children and adults, and other cases induced by B1, B2 and B4 are also distributed sporadically. The Coxsackievirus B belongs to enteroviruses, which can lead to a variety of diseases including myocarditis, aseptic meningitis, etc. At present, effective anti-Coxsackievirus B drugs have not been found clinically.

Current therapies for the Coxsackievirus B are mainly supportive and have certain limitations. For example, an early intravenous immunoglobin (IVIG) therapy may have a certain impact on survival. In addition, in terms of prevention, a recombinant Coxsackievirus vector expressing interferon-γ (IFN-γ) can play the role of a vaccine at a laboratory stage. Regarding drugs, current studies show that drugs approved by FDA (such as N-acetylcysteine as an antioxidant, orlistat as an anti-obesity, and lithium chloride as an antimicrobial drug), natural products (such as flavonoid compounds, terpenoids, alkaloids, and glycosides) that may have antiviral activity, pharmacologically active compounds (such as 9-arylpurine), and others (such as nucleoside analogs) are mainly used for CVB3. Effects of emodin and umifenovir on CVB4, effects of fluoxetine on CVB2-4 and effects of continuous alternate administration (CAA) on CVB1 have all been demonstrated in recent investigations, and rare findings on CVB5-6 have also been reported. All of the above preparations can only be recognized as lead compounds at present, rather than drugs approved for clinical use. Therefore, as prompted by the lack of marketed anti-CVBs drugs, more potential lead compounds are explored to increase the possibility of becoming marketed drugs.

Doxepin hydrochloride is an effective tricyclic antidepressant for treatment of depression and anxiety, and recently, an oral preparation of the doxepin hydrochloride has also been approved for treatment of insomnia and itching. Some recent reports prove that the doxepin hydrochloride may be associated with treatment of neuropathic pain and Parkinson's disease. A patent CN200910237043.0 discloses a pharmaceutical composition resisting herpesvirus infection, which includes, by weight percent, 5% to 15% of n-docosanol and 0.1% 10% of a non-steroidal anti-inflammatory drug, where the non-steroidal anti-inflammatory drug is selected from ibuprofen piconol, bufexamac, butyl flufenamate, doxepin hydrochloride, or indomethacin. A patent CN201811276456.5 discloses a preparation method and use of a nanocarrier. An active component included in the nanomaterial can be selected from doxepin hydrochloride, and skin diseases for which the nanocarrier is used include viral skin diseases, etc. However, up to now, relevant studies on use of doxepin hydrochloride in preparation of anti-Coxsackievirus B drugs in the prior art are not found.

### SUMMARY

Aiming at the problems of the prior art, the present disclosure provides use of doxepin hydrochloride in preparation of an antiviral drug, which provides a foundation and a basis for use of the doxepin hydrochloride in treatment of Coxsackievirus B infection.

In order to achieve the above purpose, the present disclosure adopts the following technical schemes.

The present disclosure provides use of doxepin hydrochloride in preparation of an anti-Coxsackievirus B drug.

Further, the Coxsackievirus B includes a Coxsackievirus B1 (CVB1), a Coxsackievirus B2 (CVB2), a Coxsackievirus B3 (CVB3), a Coxsackievirus B4 (CVB4), a Coxsackievirus B5 (CVB5), and/or a Coxsackievirus B6 (CVB6). Preferably, the Coxsackievirus B includes the Coxsackievirus B1 (CVB1), the Coxsackievirus B2 (CVB2), and/or the Coxsackievirus B3 (CVB3).

Generally, Coxsackieviruses can be divided into two groups, including a group A and a group B, the group A has 23 types of viruses, the group B has 6 types of viruses, and the CVB1, the CVB2, the CVB3, the CVB4, the CVB5 and the CVB6 are the 6 types of viruses in the group B.

The present disclosure further provides an anti-Coxsackievirus B drug, which includes doxepin hydrochloride and a medically acceptable auxiliary material.

Further, the medically acceptable auxiliary material includes, but is not limited to, one or more of a diluent, an absorbent, a wetting agent, an adhesive, a disintegrant, a lubricant, a colorant, and a solvent.

Further, the diluent includes, but is not limited to, one or more of starch, dextrin, sucrose, lactose, and microcrystalline cellulose.

Further, the diluent includes, but is not limited to, one or more of calcium sulfate, calcium bisulfate, calcium carbonate, and light calcium oxide.

Further, the wetting agent includes, but is not limited to, water and/or ethanol.

Further, the disintegrant includes, but is not limited to, one or more of dry starch, sodium hydroxymethyl starch, an effervescent disintegrant, and crospovidone.

Further, the lubricant includes, but is not limited to, one or more of magnesium stearate, talc powder, hydrogenated vegetable oil, and superfine silica powder.

Further, the colorant includes, but is not limited to, one or more of titanium dioxide, methylene blue, and sunset yellow.

Further, a dosage form of the anti-Coxsackievirus B drug includes a tablet, a liquid, a capsule, a powder, a suppository, and a granule.

Preferably, the dosage form of the anti-Coxsackievirus B drug is the tablet.

Further, a concentration of the doxepin hydrochloride in the anti-Coxsackievirus B drug is in a range of 3.125-90 μM.

Further, the concentration of the doxepin hydrochloride in the anti-Coxsackievirus B drug is in a range of 3.125-50 μM.

Further preferably, the concentration of the doxepin hydrochloride in the anti-Coxsackievirus B drug is in a range of 12.5-50 μM.

Furthermore preferably, the concentration of the doxepin hydrochloride in the anti-Coxsackievirus B drug is specifically 50 μM.

The present disclosure further provides a preparation method for the anti-Coxsackievirus B drug, which includes the following step: mixing the doxepin hydrochloride with the medically acceptable auxiliary material.

The present disclosure further provides use of doxepin hydrochloride in treatment of Coxsackievirus B infection.

The present disclosure further provides use of doxepin hydrochloride in treatment of aseptic meningitis, pleuralgia, panuveitis, acute pancreatitis, severe hepatitis, hand-foot-and-mouth disease, and myocarditis.

Technical effects achieved by the present disclosure are as follows.

In the present disclosure, it is found through an antiviral activity experiment based on a cytopathic effect (CPE) in vitro that doxepin hydrochloride has anti-Coxsackievirus B activity. The doxepin hydrochloride exhibits strong antiviral activity against Coxsackievirus B type 1, type 2 and type 3 and has a 50% minimal inhibitory concentration (IC₅₀) value of 10.12±0.85 μM. However, the doxepin hydrochloride does not have any antiviral activity against other enteroviruses, including enterovirus type 71 (BrCr/C4 subtype) and Coxsackievirus A (6/10/16 subtype). In addition, the present disclosure also finds that the doxepin hydrochloride inhibits virus replication at an early stage of an infection cycle, rather than affecting an entry or assembly process. In addition, some genes potentially associated with a mechanism are found through gene association network analysis. These findings provide a foundation and a basis for use of the doxepin hydrochloride in treatment of Coxsackievirus B infection.

### BRIEF DESCRIPTION OF THE DRAWINGS

FIG. 1 shows a chemical structural formula of doxepin hydrochloride (C₁₉H₂₂ClNO);

FIG. 2 shows a schematic diagram of antiviral activity of doxepin hydrochloride against CVB1 at low, medium and high titers;

FIG. 3 shows a schematic diagram of an inhibition rate of doxepin hydrochloride at a concentration of 25 μM against six virus strains;

FIG. 4 shows a schematic diagram of a plaque reduction assay of doxepin hydrochloride against a CVB1 virus, where a of FIG. 4 shows plaque images of the plaque reduction assay of two doxepin hydrochloride administration approaches against the CVB1 virus, b of FIG. 4 shows a statistical diagram of results of the plaque reduction assay of the two doxepin hydrochloride administration approaches against the CVB1 virus, and c of FIG. 4 shows experimental results of doxepin hydrochloride in inhibiting the generation of progeny viruses of the CVB1 virus;

FIG. 5 shows a schematic diagram of a time process test, where a of FIG. 5 shows a schematic diagram of administration times of doxepin hydrochloride after virus infection of cells, and b of FIG. 5 shows effects of different administration times on the generation of progeny viruses of a virus; and

FIG. 6 shows a schematic diagram of target prediction and gene association network analysis.

### DETAILED DESCRIPTION OF THE EMBODIMENTS

Embodiments of the present disclosure are described below through particular specific examples, and other advantages and effects of the present disclosure can be easily understood by persons skilled in the art according to contents disclosed in the specification. The present disclosure may also be implemented or applied through other different specific embodiments, and various modifications or changes of various details in the specification may also be made without departing from the spirit of the present disclosure on the basis of different viewpoints and applications.

Before further describing the specific embodiments of the present disclosure, it should be understood that the scope of protection of the present disclosure is not limited to the following specific embodiments. It should also be understood that terms used in the examples of the present disclosure are intended to describe specific embodiments, rather than to limit the scope of protection of the present disclosure.

When numerical ranges are given in the examples, it should be understood that, unless otherwise specified in the present disclosure, two endpoints of each numerical range and any one value between the two endpoints may be selected. Unless otherwise defined, all technical terms and scientific terms used herein have same meanings as those generally understood by persons of ordinary skill in the art to which the present disclosure belongs.

It is worth noting that all raw materials used in the present disclosure are common commercially available products and thus have no specific limitations in sources.

### Example 1

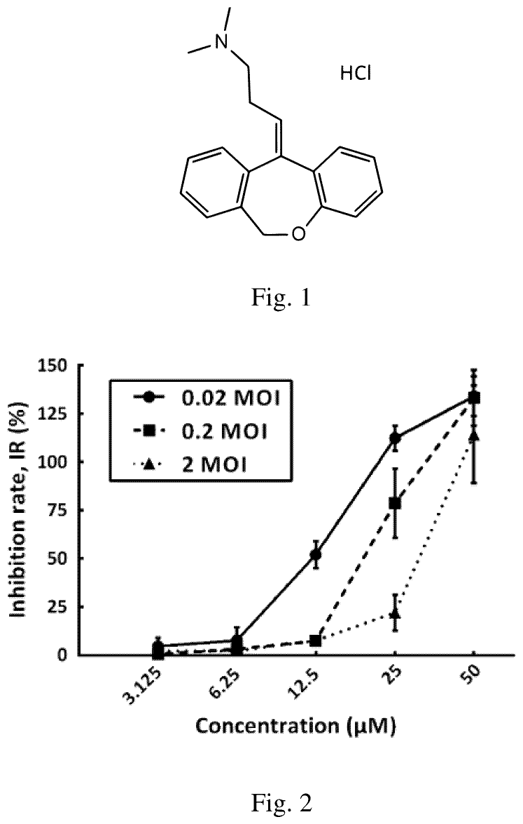

A chemical structural formula of doxepin hydrochloride is shown in FIG. 1.

Test 1: Different concentrations (50 μM, 25 μM, 12.5 μM, 6.25 μM, or 3.125 μM) of doxepin hydrochloride were added to Vero cells (cell quantity: 1.5×10⁴ cells per well) infected with 0.02 MOI, 0.2 MOI, or 2 MOI of CVB1, respectively. 72 hours later, the degree of a cytopathic effect was detected by an AlamarBlue fluorescent reagent, and an inhibition rate was calculated by using a formula. As shown in FIG. 2, each point is a mean value, and an error bar represents a standard deviation of three parallel tests.

### Test 2:

A cytotoxicity test of CVB1, CVB2 and CVB3 on Vero cells was carried out with three groups of parallel tests, respectively, and Table 1 was obtained.

TABLE 1
Anti-CVB1 activity, anti-CVB2 activity, anti-CVB3 activity,
cytotoxicity and selectivity index of doxepin hydrochloride

| Virus | CC₅₀^{a} (μM) | IC₅₀^{b} (μM) | SI^{c} |
|---|---|---|---|
| CVB1 | 90.71 ± 16.39 | 10.12 ± 0.85 | 8.96 |
| CVB2 | 90.71 ± 16.39 | 19.83 ± 8.78 | 4.57 |
| CVB3 | 90.71 ± 16.39 | 20.34 ± 5.70 | 4.46 |

Note:
In the table, ^{a}is CC₅₀ (50% cytotoxicity concentration) detected in the Vero cytotoxicity test, which is meantstandard deviation calculated in the three groups of parallel tests;
^{b}is mean ± standard deviation of IC₅₀ (50% inhibitory concentration);
and ^{c}is a selectivity index, which is a ratio of the CC₅₀ to the IC₅₀.

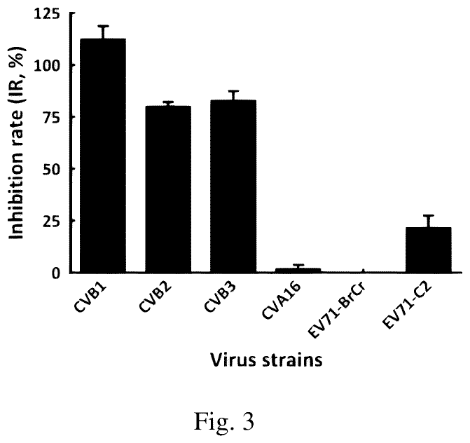

Test 3: In vitro anti-CVB1, anti-CVB2, anti-CVB3, anti-CVA16, anti-EV71 BrCr and anti-EV71-C2 virus models of Vero cells (cell quantity: 1.5×10⁴ cells per well) were established, respectively. The infected cells were treated with doxepin hydrochloride at a concentration of 25 μM for 72 hours. An inhibition rate and a standard deviation were measured by an AlamarBlue test in three parallel tests. As shown in FIG. 3, the test shows that the doxepin hydrochloride has broad-spectrum activity against Coxsackievirus B.

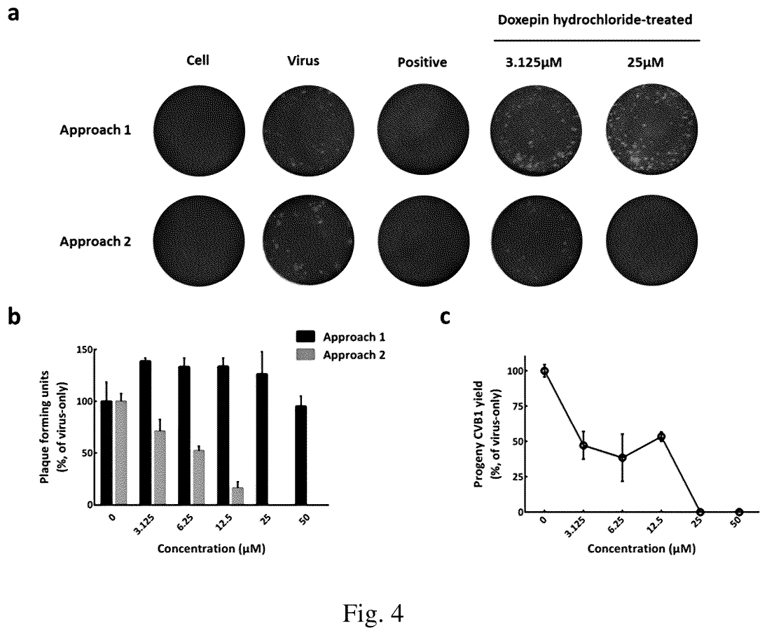

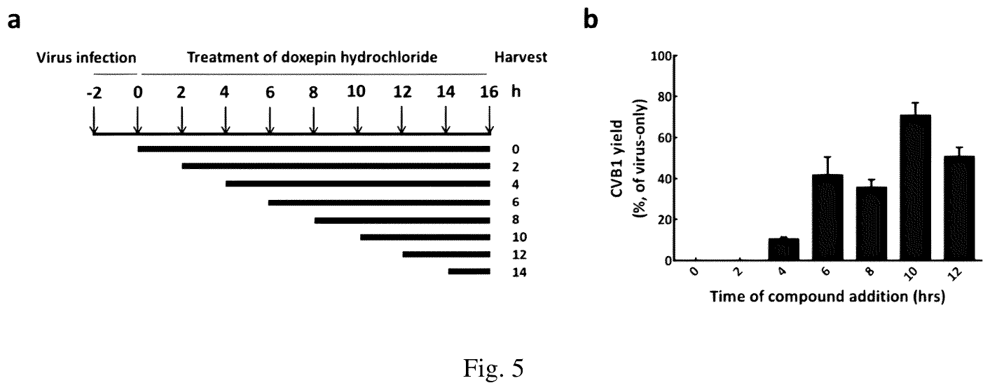

Test 4: As shown in a in FIG. 4, Vero cells were inoculated into a 24-well culture plate with a cell quantity of 2.0×10⁵ cells per well.

Method 1: Continuous gradient concentrations of doxepin hydrochloride and 30-60 PFU/well of a CVB1 virus solution were simultaneously added to a Vero cell monolayer for mixed culture for 2 hours, and then the monolayer was replaced with a drug-free overlay culture medium containing 2% of FBS and 1% of methylcellulose for continued culture for 72 hours. Method 2: The Vero cells were first added to a CVB1 virus solution, and 2 hours later, the virus solution was replaced with an overlay culture medium containing gradient concentrations of a drug for continued culture for 72 hours. Formed plaques were fixed with 10% formaldehyde and stained with 1% crystal violet. In the figure, Positive represents that penduletin was used as a positive control group.

As shown in b in FIG. 4, a plaque forming unit was calculated through three parallel tests, and only a CVB1 group was considered as having 100% plaque formation.

As shown in c in FIG. 4, the doxepin hydrochloride inhibited the progeny yield of CVB1. The Vero cells were infected with 0.02 MOI of CVB1 and simultaneously treated with a corresponding concentration of doxepin hydrochloride. After culture for 72 hours, a supernatant was titrated by a plaque reduction test, and results were obtained from three groups of parallel tests.

Test 5: In FIG. 5, a shows a schematic diagram of administration times of doxepin hydrochloride after virus infection of cells. b shows effects of different administration times on the generation of progeny viruses of a virus, that is, a schematic diagram of conditions of doxepin hydrochloride in inhibiting replication of CVB1 at different time points. Only a virus group had a plaque forming rate of 100%, and results were calculated through three groups of parallel tests.

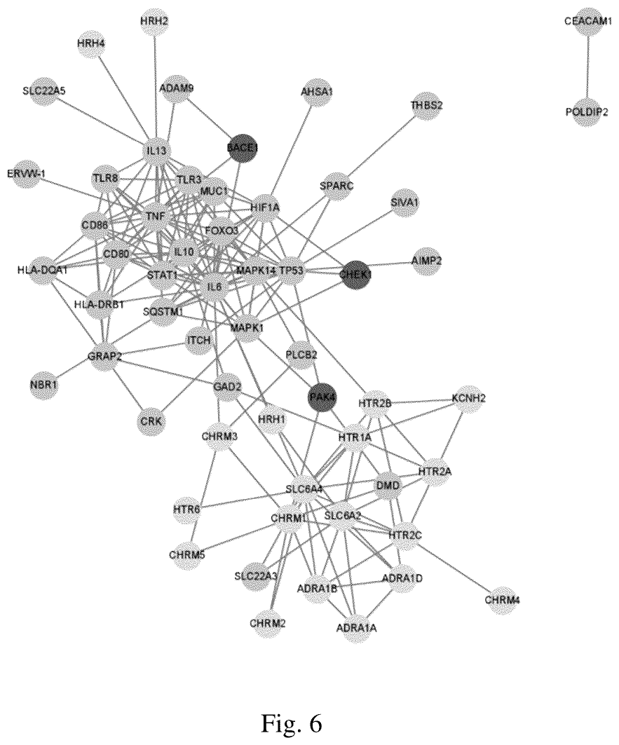

Test 6: As shown in FIG. 6, highlighted black nodes were core targets of doxepin hydrochloride predicted based on pharmacophore similarity. Other background networks were composed of antidepressant targets of doxepin hydrochloride and genes associated with Coxsackievirus B infection obtained from a DisGeNET database.

Finally, it should be noted that the above contents are merely used for illustrating the technical schemes of the present disclosure and are not intended to limit the scope of protection of the present disclosure, and all simple modifications or equivalent substitutions of the technical schemes of the present disclosure that are made by persons of ordinary skill in the art are not departed from the essence and scope of the technical schemes of the present disclosure.

1. Use of doxepin hydrochloride in preparation of an anti-Coxsackievirus B drug.

2. The use according to claim 1, wherein the Coxsackievirus B comprises a Coxsackievirus B1, a Coxsackievirus B2, a Coxsackievirus B3, a Coxsackievirus B4, a Coxsackievirus B5, and/or a Coxsackievirus B6.

3. The use according to claim 1, wherein the Coxsackievirus B comprises a Coxsackievirus B1, a Coxsackievirus B2, and/or a Coxsackievirus B3.

4. An anti-Coxsackievirus B drug, comprising doxepin hydrochloride and a medically acceptable auxiliary material.

5. The anti-Coxsackievirus B drug according to claim 4, wherein a dosage form of the anti-Coxsackievirus B drug comprises a tablet, a liquid, a capsule, a powder, a suppository, and a granule.

6. The anti-Coxsackievirus B drug according to claim 4, wherein a dosage form of the anti-Coxsackievirus B drug is a tablet.

7. The anti-Coxsackievirus B drug according to claim 4, wherein a concentration of the doxepin hydrochloride in the anti-Coxsackievirus B drug is in a range of 3.125-90 μM.

8. The anti-Coxsackievirus B drug according to claim 7, wherein the concentration of the doxepin hydrochloride in the anti-Coxsackievirus B drug is in a range of 3.125-50 μM.

9. The anti-Coxsackievirus B drug according to claim 8, wherein the concentration of the doxepin hydrochloride in the anti-Coxsackievirus B drug is in a range of 12.5-50 μM.

10. A preparation method for the anti-Coxsackievirus B drug according to claim 1, comprising the following step: mixing the doxepin hydrochloride with the medically acceptable auxiliary material.

11. Use of doxepin hydrochloride in treatment of Coxsackievirus B infection.

12. Use of doxepin hydrochloride in treatment of aseptic meningitis, pleuralgia, panuveitis, acute pancreatitis, severe hepatitis, hand-foot-and-mouth disease, and myocarditis.
